(19) United States
(12) Patent Application Publication
(10) Pub. No.: US 2026/0100247 A1
(43) Pub. Date: Apr. 9, 2026

(54) SYSTEMS AND METHODS FOR PROCESSING CLINICO-GENOMIC DATA

(71) Applicant: Foundation Medicine, Inc., Boston, MA (US)

(72) Inventors: Gerald LI, Cambridge, MA (US); Ryon P. GRAF, Encinitas, CA (US)

(73) Assignee: Foundation Medicine, Inc., Boston, MA (US)

(21) Appl. No.: 19/115,680

(22) PCT Filed: Sep. 29, 2023
(86) PCT No.: PCT/US2023/075524

§ 371 Date: Mar. 26, 2025

Related U.S. Application Data
(60) Provisional application No. 63/411,905, filed on Sep. 30, 2022.

(51) Int. Cl. G16B 20/10; G16B 20/20; G16B 40/20; G16H 50/80

(52) U.S. Cl. CPC G16B 20/10; G16B 20/20; G16B 40/20; G16H 50/80

(57) ABSTRACT

Methods for processing clinico-genotnic data are described. The methods may comprise, for example, receiving, at one or more processors, input data that specifies a plurality of prognostic features for a disease; extracting, using the one or more processors, a first data set comprising data corresponding to the plurality of prognostic features for the disease from a clinico-genomic database; generating, using the one or more processors, a second data set based on the first data set, wherein the second data set comprises data from the first data set, data derived from data in the first data set, or a combination thereof; and storing using the one or more processors, the second data set in a standardized table format. In one or more embodiments, the methods further comprise outputting the second data set in the standardized table format.

### CROSS-REFERENCE TO RELATED APPLICATIONS

This application claims the priority benefit of U.S. Provisional Patent Application Ser. No. 63/411,905, filed Sep. 30, 2022, the contents of which are incorporated herein by reference in their entirety.

### FIELD OF DISCLOSURE

The present disclosure relates generally to methods and systems for analyzing genomic profiling data, and more specifically to methods and systems for processing clinico-genomic data into a standardized format.

### BACKGROUND

Clinico-genomic data are complex, vast, and not standardized. For example, clinico-genomic data may be derived from multiple sources and comprise a range of formats. Accordingly, the parsing of clinico-genomic data into meaningful information is formidable. This parsing often requires writing and rewriting bespoke scripts for each data set, and the process is laborious and inefficient. Improved methods for handling clinico-genomic data is needed. The present disclosure addresses these needs.

### BRIEF SUMMARY

The embodiments of the present disclosure are provided to address the complex data structure of one or more databases, e, g,, a Clinico-genomic Database (CGDB). For example, the data in the CGDB may not be standardized and may be found across many tables. Performing even the same data wrangling steps and obtaining data across these many data sets may consume considerable time and effort for each new analysis. Embodiments of the present disclosure aim to standardize these initial data manipulation steps via an algorithm that generates a standardized data set from the CGDB database. In one or more examples, the standardized data set may include one or more tables in a standardized format. These standardized tables may be referred to as intermediate tables herein.

Embodiments of the present disclosure provide methods for improving the performance of the computing device by efficiently processing one or more data sets into a standardized format, so that similar or identical data wrangling steps are not repeated across multiple input data sets. At least one or more methods described in the embodiments of the present disclosure can also provide specific improvement over prior art systems by aggregating input data sets and converting them into a standardized format, regardless of the format in which the user provided the input data sets.

Embodiments of the present disclosure provide systems and methods for processing clinico-genomic data. In one or more embodiments, the methods comprise: receiving, at one or more processors, input data that specifies a plurality of prognostic features for a disease; extracting, using the one or more processors, a first data set comprising data corresponding to the plurality of prognostic features for the disease from a clinico-genomic database; generating, using the one or more processors, a second data set based on the first data set, wherein the second data set comprises data from the first data set, data derived from data in the first data set, or a combination thereof; and storing using the one or more processors, the second data set in a standardized table format. In one or more embodiments, the methods further comprise outputting the second data set in the standardized table format.

In one or more embodiments, the disease is breast cancer, colorectal cancer, endometrial cancer, gastric cancer, hepatocellular carcinoma, head and neck cancers, melanoma, non-small-cell lung cancer, ovarian cancer, pancreatic cancer, prostate cancer, renal cell carcinoma, small cell lung cancer, or urothelial cancer.

In one or more embodiments, the disease is breast cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, histology, presence of brain metastases, bone metastases, stage of diagnosis, menopausal status, visceral crisis, ER status, PR status, HER2 status, PD-L1 immunohistochemistry, germline BRCA status, PI3CA status, or any combination thereof.

In one or more embodiments, the disease is colorectal cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, colorectal site (including sidedness for colon cancer), stage of diagnosis, BRAF mutation status, RAS mutation status, dMMR/MSI, HER2 status, consensus molecular subtypes, platelets status, or any combination thereof.

In one or more embodiments, the disease is endometrial cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, histology, grade, ER status, PR status, HER2 status, TCGA subgroup, POLE status, MSI-H/dMMR status, TP53 status, presence of brain metastases, presence of metastases above a diaphragm, disease stage at diagnosis, beta-catenin alteration status, serum CA-125 level, history of endometriosis, BMI, residual disease in abdomen after primary surgery, blood pressure, or any combination thereof.

In one or more embodiments, the disease is gastric cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, tumor type/disease site, disease stage at diagnosis, Siewert classification, smoking status, anemia, H. pylori status, alcohol use, EBV, surgery, HER2 status, PD-L1 status, MSI/MMR status, family history, or any combination thereof.

In one or more embodiments, the disease is hepatocellular carcinoma, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, disease stage at diagnosis, Child-Pugh score, encephalopathy, ascites, bilirubin status, primary biliary cirrhosis status, aspartate transaminase status, alanine transaminase status, albumin (quantitative), prothrombin time (PT), international normalized ratio (INR), blood urea nitrogen (BUN), complete blood count (CBC), platelets status, alpha fetoprotein (AFP) status, ALBI grade, microsatellite instability (MSI) status, mismatch repair (MMR) status, tumor mutational burden (TMB), or any combination thereof.

In one or more embodiments, the disease is a head and neck cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, primary site, disease stage at diagnosis, smoking status, HPV/p16 status, alcohol use, Epstein-Barr virus (EBV) status, surgery status, radiotherapy status, PD-L1 status, or any combination thereof.

In one or more embodiments, the disease is melanoma, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, sites of metastases, BRAF V600 mutation status, KIT status, NRAS status, NTRK status, Breslow thickness, ulceration, mitotic rate, tumor location (axial vs extremity), lymphovascular invasion, microsatellites (local spread), NF1, prednisone, or any combination thereof.

In one or more embodiments, the disease is non-small-cell lung cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrdrogenase level, neutrophil to lymphocyte ratio, histology, smoking history, presence of brain metastases, bone metastases, disease stage at diagnosis, EGFR mutation status, ALK rearrangement status, ROS1 rearrangement status, BRAF mutation status, KRAS mutation status, MET exon 14 skipping mutation status, RET rearrangement status, NTRK rearrangement status, MET amplification status, ERBB2 mutation status, PD-L1 immunohistochemistry, TMB, or any combination thereof.

In one or more embodiments, the disease is ovarian cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, histology, disease stage at diagnosis, disease grade at diagnosis, HRD status, CA-125 status, TP53 status, gabapentin status, surgical removal of macroscopic disease (R0) status, platinum sensitivity, neoadjuvant chemotherapy vs surgery as an initial intervention, family history, germline BRCA status, BRCA1 status, BRCA2 status, RAD51C status, RAD51D status, BARD1 status, BRIP1 status, PALB2 status, MLH1 status, MSH2 status, MSH6 status, PMS2 status, STK11 status, or any combination thereof.

In one or more embodiments, the disease is pancreatic cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, high neutrophil to lymphocyte ratio, disease stage at diagnosis, radiotherapy, cancer antigen 19-9 status, MSI status, MMR status, TMB, prior therapies, BRCA mutation status, PALB2 mutation status, ALK fusion status, NRG1 fusion status, NTRK fusion status, ROS1 fusion status, BRAF mutation status, HER2 mutation status, KRAS mutation status, or any combination thereof.

In one or more embodiments, the disease is prostate cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrdrogenase level, neutrophil to lymphocyte ratio, number of bone metastases, liver metastases present, PSA level, years since original PCA diagnosis, prior 2^{nd} generation novel hormonal therapy, prior taxane, small cell histology, PSA doubling time/PSA velocity, recent development of new lesions, CTC count, ctDNA fraction, bone alkaline phosphatase level, presence of N-telopeptides in urine, extent of bone involvement, patient mobility, patient ability to climb stairs, insulin use, antithrombotic use, antiarrhythmic agent use, or any combination thereof.

In one or more embodiments, the disease is renal cell carcinoma and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, histology, disease stage at diagnosis, International Metastatic RCC Database Consortium (IMDC) risk score, recent diagnosis of metastases, recurrence of metastases within 1 year vs recurrence after 1 year, hypercalcemia, neutrophil status, platelet status, anemia, c-reactive protein level, inflammation, IL-6 level, IL-8 level, HGF hepatocyte growth factor level, osteopontin level, BAP1 level, PBRM1 level, 3p loss, 5q gain, 7q gain, 8p loss, 9p loss, 14q loss, or any combination thereof.

In one or more embodiments, the disease is small cell lung cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, SCLC stage at diagnosis, disease stage at diagnosis, smoking status, brain metastases, localized symptomatic sites, resection status, radiotherapy status, prior immunotherapy status, or any combination thereof.

In one or more embodiments, the disease is urothelial cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, primary site, disease stage at diagnosis, disease grade at diagnosis, CBC, asparate aminotransferase level, alanine aminotransferase level, bilirubin level, glomerular filtration rate, PD-L1 status, fibroblast growth factor receptor (FGFR) status, De Ritis ratio, or any combination thereof.

In one or more embodiments, the data in the second data set that is derived from data in the first data set comprises a patient's age at a start of a line of therapy, a determination of whether the patient's albumin levels are less than a lower limit of normal, a determination of whether the patient's alkaline phosphatase levels are greater than an upper limit of normal, a determination of whether the patient's serum creatinine levels are greater than an upper limit of normal, a determination of the patient's pooled ECOG value, a determination of whether the patient should be excluded when applying a 90 day gap rule, a determination of whether the patient's hemoglobin levels are less than a lower limit of normal, a determination of whether the patient's line of therapy has had a maintenance line rolled in, a determination of whether the patient's lactate dehydrogenase levels are greater than an upper limit of normal, a determination of a numerical value for a neutrophil-to-lymphocyte ratio, a determination of whether the neutrophil-to-lymphocyte ratio is greater than 2.5, a determination of whether the patient has evidence of having received opioid pain medication in a period of 62 days preceding the start of the line of therapy, a determination of an end date used in a calculation of the patient's overall survival (OS), a determination of a time to death or censoring for the patient's OS analysis, a determination of an entry date used in the calculation of the patient's OS, a determination of the patient's delayed entry time in months, a determination of whether the end date was an event or censor for OS analysis, a determination of whether the patient has evidence of having received steroid medication in a period of 62 days preceding the start date of their line of therapy, a determination of whether the patient's time to discontinuation (TTD) was an event or censor, a determination of TTD in months for TTD analysis, a determination of whether the patient's time to next treatment (TTNT) was an event or censor, a determination of TTNT in months for TTNT analysis, disease-free survival (DFS), time-to-treatment failure (TTF), durable complete response (DCR), or any combination thereof.

In one or more embodiments, the data in the second data set that is derived from data in the first data set comprises a pre-computed endpoint for a survival analysis or a time-to-event analysis.

In one or more embodiments, the pre-computed endpoint for survival analysis comprises time from start of any line of treatment of the disease to a time of death, overall survival (OS), progression free survival (PFS), objective response rate (ORR), time-to-tumor progression (TTP), time-to-next treatment (TTNT), disease-free survival (DFS), time-to-tumor progression (TTP), time-to-treatment failure (TTF), or durable complete response (DCR).

In one or more embodiments, the standardized table format comprises a matrix in which each row specifies a line of therapy, and each column comprises data for a category of prognostic and analytically useful variable.

In one or more embodiments, the clinico-genomic database can comprise a single entity or a joint collaboration clinico-genomic database (CGDB).

In one or more embodiments, an analysis of clinico-genomic data comprises: receiving, at one or more processors, a first input from a user, wherein the first input specifies a disease; accessing, using the one or more processors, a standardized table of clinico-genomic data for the disease; receiving, at the one or more processors, at least a second input from the user; performing, using the one or more processors, an analysis based on the at least second input and the clinico-genomic data included in the standardized table; and outputting, using the one or more processors, a result of the analysis of the clinico-genomic data included in the standardized table.

In one or more embodiments, the method further comprises accessing additional data from a clinico-genomic database and using the additional data as part of the analysis.

In one or more embodiments, the analysis comprises a Kaplan Meier survival analysis or a log rank test.

In one or more embodiments, the analysis comprises a statistical regression analysis.

In one or more embodiments, the statistical regression analysis comprises a Cox proportional hazards regression analysis.

In one or more embodiments, a system comprises: one or more processors; and a memory communicatively coupled to the one or more processors and configured to store instructions that, when executed by the one or more processors, cause the system to: receive, at one or more processors, input data that specifies a plurality of prognostic features for a disease; extract, using the one or more processors, a first data set comprising data corresponding to the plurality of prognostic features for the disease from a clinico-genomic database; generate, using the one or more processors, a second data set based on the first data set, wherein the second data set comprises data from the first data set, data derived from data in the first data set, or a combination thereof; and store, using the one or more processors, the second data set in a standardized table format.

In one or more embodiments, a non-transitory computer-readable storage medium stores one or more programs, the one or more programs comprises instructions, which when executed by one or more processors of a system, cause the system to: receive, at one or more processors, input data that specifies a plurality of prognostic features for a disease; extract, using the one or more processors, a first data set comprising data corresponding to the plurality of prognostic features for the disease from a clinico-genomic database; generate, using the one or more processors, a second data set based on the first data set, wherein the second data set comprises data from the first data set, data derived from data in the first data set, or a combination thereof; and store, using the one or more processors, the second data set in a standardized table format.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 depicts a non-limiting example of an exemplary schematic showing a process for performing an analysis of clinico-genomic data, in accordance with some embodiments of the present disclosure.

FIG. 2 depicts a non-limiting example of an intermediate table, in accordance with some embodiments of the present disclosure.

FIG. 3A depicts a non-limiting example of supplementary data, in accordance with some embodiments of the present disclosure.

FIG. 3B depicts a non-limiting example of supplementary data, in accordance with some embodiments of the present disclosure.

FIG. 4 depicts an exemplary method for processing clinico-genomic data, in accordance with some embodiments of the present disclosure.

FIG. 5 depicts an exemplary computing device or system in accordance with some embodiments of the present disclosure.

FIG. 6 depicts an exemplary computer system or computer network, in accordance with some instances of the systems described herein.

### DETAILED DESCRIPTION

The embodiments of the present disclosure are provided to address the complex data structure of one or more databases, e.g., a Clinico-genomic Database (CGDB). For example, the data in the CGDB may not be standardized and may be found across many tables. Performing even the same data wrangling steps and obtaining data across these many data sets may consume considerable time and effort for each new analysis. Embodiments of the present disclosure aim to standardize these initial data manipulation steps via an algorithm that generates a standardized data set from the CGDB database. In one or more examples, the standardized data set may include one or more tables in a standardized format. These standardized tables may be referred to as intermediate tables herein.

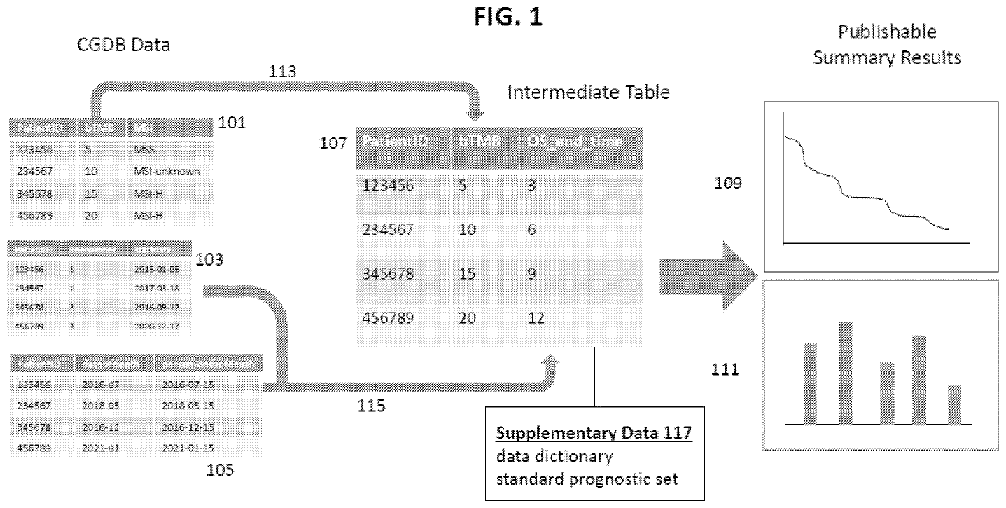

FIG. 1, illustrates an exemplary schematic showing a process for performing an analysis of clinico-genomic data to generate a standardized table according to embodiments of the present disclosure. Fields from the CGDB database, the CGDB database comprising one or more tables, can be duplicated or transformed into fields in the intermediate table. The intermediate table can then be used to quickly produce publishable figures. In one or more examples, the CGDB can comprise a single entity or a joint collaboration clinico-genomic database.

As shown in the figure, sample and disparate CGDB data are found across multiple tables, e.g., tables 101, 103, and 105. In one or more examples, each table may not comprise an identical set of fields. The data sets included in tables 101, 103, and 105 may be manipulated via one or more data manipulation algorithms 113 and 115. As shown in FIG. 1, data manipulation algorithm 113 can duplicate a field and its respective data from table 101 to intermediate table 107. In one or more examples, each intermediate table 107 may be associated with supplementary data 117. Data manipulation algorithm 115 can use one or more fields from table 103 and table 105 to derive a related field in intermediate table 107. In other words, various data manipulation algorithms may be used to generate the intermediate table from the data across multiple non-uniform tables, e, g,, by duplicating data from a field of a first table to an intermediate table field and/or deriving a new field from one or more fields across one or more tables. For example, a derived field in intermediate table 107 can include a determination of whether a patient should be excluded when applying a 90 day gap rule, which is a rule that can be used as a data completeness check.

The intermediate table 107 can be used to conduct one or more types of analyses. In one or more examples, the analysis can include, but is not limited to a Kaplan-Meier survival analysis, log rank test, a Cox proportional hazards regression analysis, or some other regression analysis. For example, from the intermediate table 107, publishable summary graphs, such as those graphs represented in 109 and 111, can be readily produced. In one or more examples, the analysis can be based on supplemental data (e.g., supplementary data 117). While a single intermediate table 107 is shown in FIG. 1, a skilled artisan will understand that multiple intermediate tables may be generated based on the CGDB data in tables 101, 103, and 105.

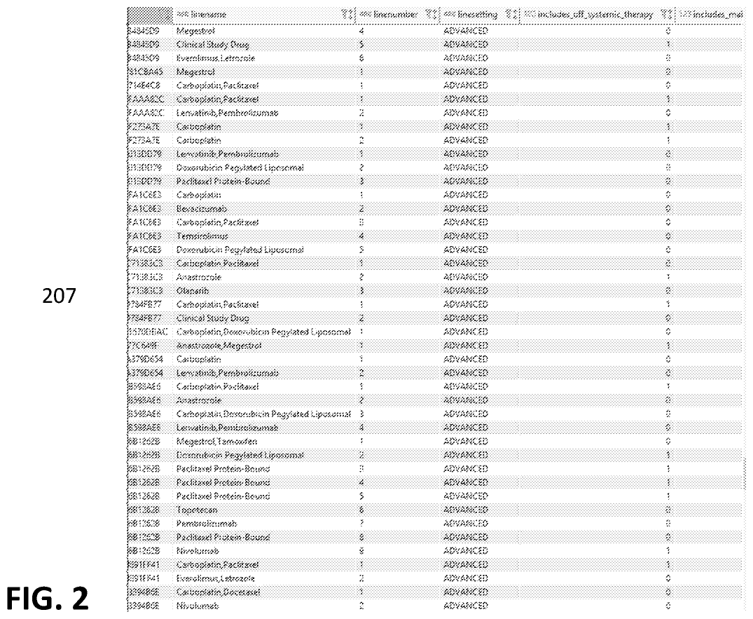

FIG. 2 illustrates an exemplary intermediate table in accordance with embodiments of the present disclosure. As shown in the figure, intermediate table 207 can include one or more rows and one or more columns. In one or more examples, each row in intermediate table 207 corresponds an observed line of therapy for one or more patients. If a patient has received multiple lines of therapy, the intermediate table 207 may include multiple rows to reflect each of these lines of therapy. In one or more examples, each column in the intermediate table 207 may include a prognostic or analytically useful variable. As used herein, the columns of the intermediate table 207 may be referred to as fields.

In some instances, each intermediate table (e.g., intermediate table 107, 207) can comprise data for a single disease, such as breast cancer. In one or more examples, the disease may include but is not limited to breast cancer, colorectal cancer, endometrial cancer, gastric cancer, hepatocellular carcinoma, head and neck cancers, melanoma, non-small-cell lung cancer, ovarian cancer, pancreatic cancer, prostate cancer, renal cell carcinoma, small cell lung cancer, or urothelial cancer. In some instances, different intermediate tables can feature a different set of fields, based on the disease of interest. In some embodiments, the selection of the fields for each disease may be bespoke and optimized for analyzing and predicting clinical patient outcomes. For example, the fields may be selected and optimized to predict clinical patient outcomes in advanced and metastatic settings. As another example, selected fields may comprise data for a survival analysis, such as a time-to-event-analysis, wherein the time from some start time to some end time, such as death, progression, or next treatment, may be studied. Examples of diseases and associated fields or prognostic features are provided in the examples section of the disclosure.

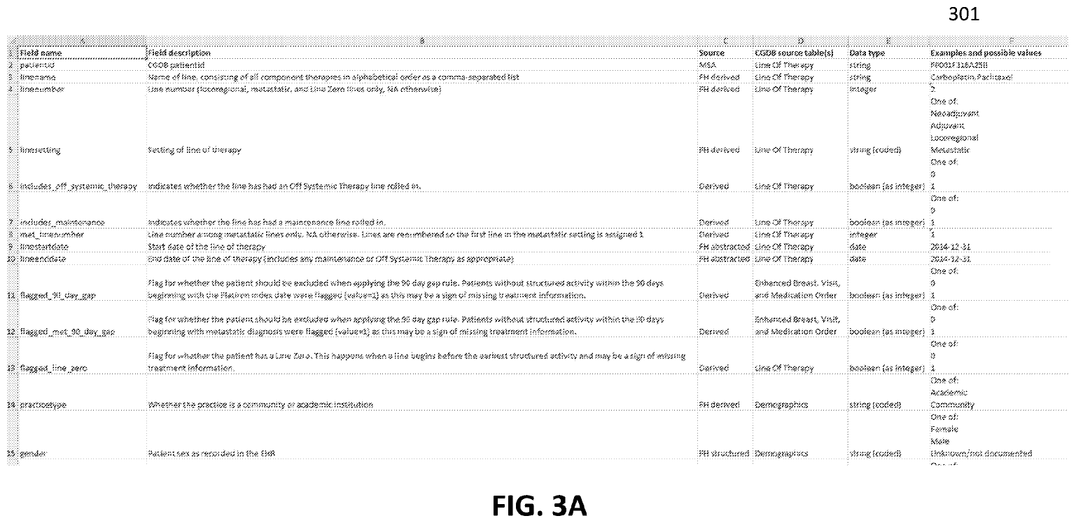

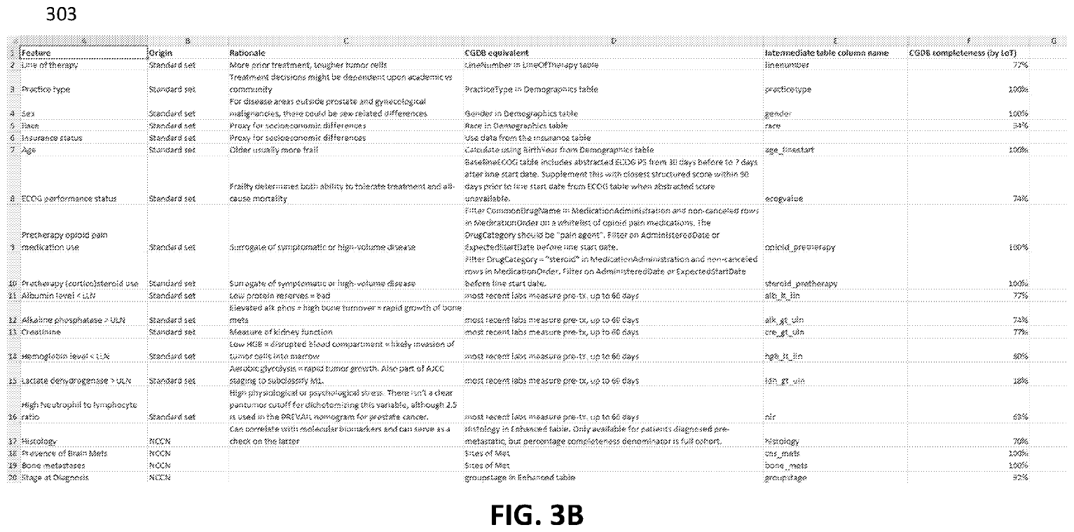

In some embodiments, an intermediate table (e.g., intermediate table 107, 207) may be associated with supplementary data (e.g., supplementary data 117). The supplementary data can include, for example, a data dictionary, and a standard prognostic set. FIG. 3A illustrates an exemplary data dictionary 301 according to embodiments of the present disclosure. In one or more examples, the data dictionary 301 may correspond to metadata for the fields presented in the corresponding intermediate table. The data dictionary 301 can provide extensive documentation, including but not limited to, field descriptions, justifications for the included fields, the data types of the included fields, and how the included fields relate to the original CGDB fields. FIG. 3B illustrates a standard prognostic set 303 according to embodiments of the present disclosure. The standard prognostic 303 set may provide further supplemental data for the intermediate table, including but not limited to, literature reviews that support the justifications for the included fields. In some examples, the standard prognostic set 303 may include potential field names that could be useful for future incorporation into the intermediate table.

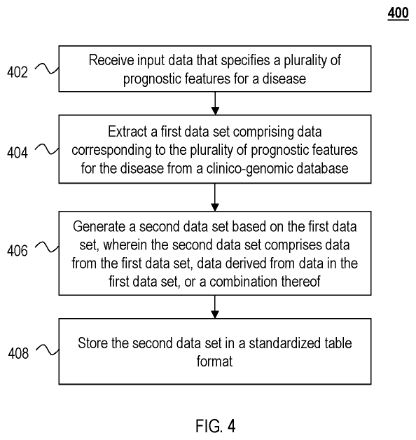

FIG. 4 provides a non-limiting example of a method for processing clinico-genomic data, in accordance with some embodiments of the present disclosure.

Process 400 can be performed, for example, using one or more electronic devices implementing a software platform. In some examples, process 400 is performed using a client-server system, and the blocks of process 400 are divided up in any manner between the server and a client device. In other examples, the blocks of process 400 are divided up between the server and multiple client devices. Thus, while portions of process 400 are described herein as being performed by particular devices of a client-server system, it will be appreciated that process 400 is not so limited. In other examples, process 400 is performed using only a client device or only multiple client devices. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail below) are exemplary by nature and, as such, should not be viewed as limiting.

At block 402 of FIG. 4, the system can receive input data that specifies a plurality of prognostic features for a disease. For example, the system can receive input data corresponding to a plurality of data sets. Referring briefly to FIG. 1, the input data may correspond to one or more tables such as tables 101, 103, and 105. As discussed above, the various fields, e.g., columns of tables 101, 103, and 105 may correspond to a plurality of prognostic features for a disease.

At block 404 of FIG. 4, the system can extract a first data set comprising data corresponding to the plurality of prognostic features for the disease from a clinico-genomic database. For example, data manipulation algorithm 113 can extract the data set corresponding to the data in table 101. As another example, data manipulation algorithm 115 can extract the data set corresponding to the data in table 103 and table 105.

At block 406 of FIG. 4, the system can generate a second data set based on the first data set. In one or more examples, the second data set (e.g., intermediate table) can comprise data from the first data set, data derived from data in the first data set, or a combination thereof. For example, a data manipulation algorithm (e.g., data manipulation algorithm 113) can generate a data set (e.g., intermediate table 107) based on the extracted data set (e.g., data in table 101). As described above, data manipulation algorithm 113 can process the data from table 101, such that the data included in the intermediate table 107 may comprise the data from table 101. In one or more examples, additional data manipulation algorithms (e.g., data manipulation algorithm 115) can further be used to generate the intermediate table. For example, data manipulation algorithm 115 can process the data from 103 and/or table 105, such that the data included in the intermediate table 107 may comprise data derived from table 103 and/or table 105).

At block 408, the system can store the second data set in a standardized table format. In one or more examples, data manipulation algorithms 113 and 115 use input data from tables 101, 103, and 105, to output and ultimately store data in intermediate table 107.

Intermediate tables may be applied to a plurality of analytical use cases. In some instances, a user may have to perform further analyses based on the intermediate table in order to apply the intermediate table to more specific applications. In one or more examples, the data in the intermediate table data can be updated regularly. In such embodiments, older versions of the intermediate table may be archived.

Embodiments of the present disclosure provide systems and methods for generating intermediate tables that can be reused for multiple analysis projects, can be shared to facilitate onboarding of new CGDB users, and can be used to drive a convergence towards best, and potentially standardized, practices. Given these advantages, intermediate tables promote greater consistency and rigorousness across disparate analyses and encourages greater analysis efficiency.

### Computer Systems and Networks

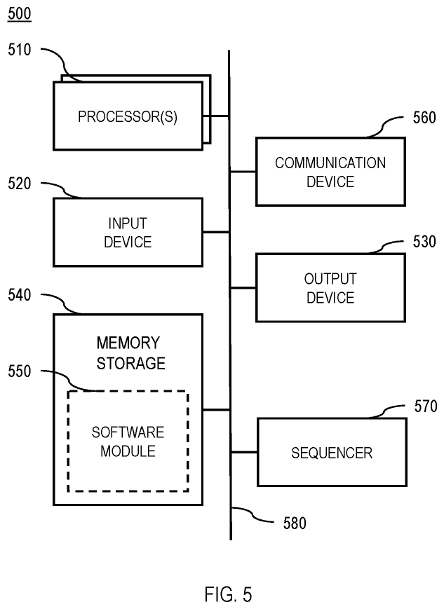

FIG. 5 illustrates an example of a computing device or system in accordance with one or more embodiments of the present disclosure. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more processor(s) 510, input devices 520, output devices 530, memory or storage devices 540, communication devices 560, and nucleic acid sequencers 570. Software 550 residing in memory or storage device 540 may comprise, e.g., an operating system as well as software for executing the methods described herein. Input device 520 and output device 530 can generally correspond to those described herein and can either be connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical system bus 580, Ethernet connection, or any other wire transfer technology) or wirelessly (e.g., Bluetooth®, Wi-Fi®, or any other wireless technology).

Software module 550, which can be stored as executable instructions in storage 540 and executed by processor(s) 510, can include, for example, an operating system and/or the processes that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

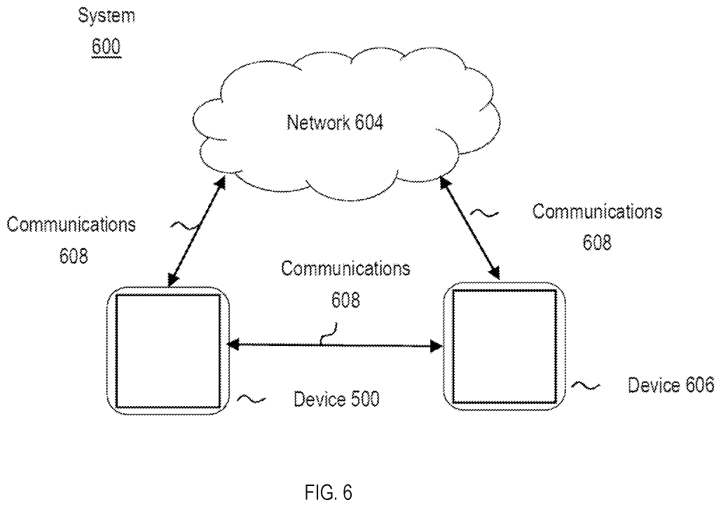

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can be implemented using any operating system, e.g., an operating system suitable for operating on the network. Software module 550 can be written in any suitable programming language, such as R, SQL, C, C++, Java, or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 606 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

### Exemplary Implementations

Exemplary implementations of the methods and systems described herein include:
1. A computer-implemented method for processing clinico-genomic data comprising:

receiving, at one or more processors, input data that specifies a plurality of prognostic features for a disease;

extracting, using the one or more processors, a first data set comprising data corresponding to the plurality of prognostic features for the disease from a clinico-genomic database;

generating, using the one or more processors, a second data set based on the first data set, wherein the second data set comprises data from the first data set, data derived from data in the first data set, or a combination thereof; and

storing, using the one or more processors, the second data set in a standardized table format.
2. The computer-implemented method of clause 1 further comprising outputting the second data set in the standardized table format.
3. The computer-implemented method of any one of clauses 1 to 2, wherein the disease is breast cancer, colorectal cancer, endometrial cancer, gastric cancer, hepatocellular carcinoma, head and neck cancers, melanoma, non-small-cell lung cancer, ovarian cancer, pancreatic cancer, prostate cancer, renal cell carcinoma, small cell lung cancer, or urothelial cancer.
4. The computer-implemented method of clause 3, wherein the disease is breast cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, histology, presence of brain metastases, bone metastases, stage of diagnosis, menopausal status, visceral crisis, ER status, PR status, HER2 status, PD-L1 immunohistochemistry, germline BRCA status, PI3CA status, or any combination thereof.
5. The computer-implemented method of clause 3, wherein the disease is colorectal cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, colorectal site (including sidedness for colon cancer), stage of diagnosis, BRAF mutation status, RAS mutation status, dMMR/MSI, HER2 status, consensus molecular subtypes, platelets status, or any combination thereof.
6. The computer-implemented method of clause 3, wherein the disease is endometrial cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, histology, grade, ER status, PR status, HER2 status, TCGA subgroup, POLE status, MSI-H/dMMR status, TP53 status, presence of brain metastases, presence of metastases above a diaphragm, disease stage at diagnosis, beta-catenin alteration status, serum CA-125 level, history of endometriosis, BMI, residual disease in abdomen after primary surgery, blood pressure, or any combination thereof.
7. The computer-implemented method of clause 3, wherein the disease is gastric cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, tumor type/disease site, disease stage at diagnosis, Siewert classification, smoking status, anemia, H. pylori status, alcohol use, EBV, surgery, HER2 status, PD-L1 status, MSI/MMR status, family history, or any combination thereof.
8. The computer-implemented method of clause 3, wherein the disease is hepatocellular carcinoma, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, disease stage at diagnosis, Child-Pugh score, encephalopathy, ascites, bilirubin status, primary biliary cirrhosis status, aspartate transaminase status, alanine transaminase status, albumin (quantitative), prothrombin time (PT), international normalized ratio (INR), blood urea nitrogen (BUN), complete blood count (CBC), platelets status, alpha fetoprotein (AFP) status, ALBI grade, microsatellite instability (MSI) status, mismatch repair (MMR) status, tumor mutational burden (TMB), or any combination thereof.
9. The computer-implemented method of clause 3, wherein the disease is a head and neck cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, primary site, disease stage at diagnosis, smoking status, HPV/p16 status, alcohol use, Epstein-Barr virus (EBV) status, surgery status, radiotherapy status, PD-L1 status, or any combination thereof.
10. The computer-implemented method of clause 3, wherein the disease is melanoma, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, sites of metastases, BRAF V600 mutation status, KIT status, NRAS status, NTRK status, Breslow thickness, ulceration, mitotic rate, tumor location (axial vs extremity), lymphovascular invasion, microsatellites (local spread), NF1, prednisone, or any combination thereof.
11. The computer-implemented method of clause 3, wherein the disease is non-small-cell lung cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrdrogenase level, neutrophil to lymphocyte ratio, histology, smoking history, presence of brain metastases, bone metastases, disease stage at diagnosis, EGFR mutation status, ALK rearrangement status, ROS1 rearrangement status, BRAF mutation status, KRAS mutation status, MET exon 14 skipping mutation status, RET rearrangement status, NTRK rearrangement status, MET amplification status, ERBB2 mutation status, PD-L1 immunohistochemistry, TMB, or any combination thereof.
12. The computer-implemented method of clause 3, wherein the disease is ovarian cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, histology, disease stage at diagnosis, disease grade at diagnosis, HRD status, CA-125 status, TP53 status, gabapentin status, surgical removal of macroscopic disease (R0) status, platinum sensitivity, neoadjuvant chemotherapy vs surgery as an initial intervention, family history, germline BRCA status, BRCA1 status, BRCA2 status, RAD51C status, RAD51D status, BARD1 status, BRIP1 status, PALB2 status, MLH1 status, MSH2 status, MSH6 status, PMS2 status, STK11 status, or any combination thereof.
13. The computer-implemented method of clause 3, wherein the disease is pancreatic cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, high neutrophil to lymphocyte ratio, disease stage at diagnosis, radiotherapy, cancer antigen 19-9 status, MSI status, MMR status, TMB, prior therapies, BRCA mutation status, PALB2 mutation status, ALK fusion status, NRG1 fusion status, NTRK fusion status, ROS1 fusion status, BRAF mutation status, HER2 mutation status, KRAS mutation status, or any combination thereof.
14. The computer-implemented method of clause 3, wherein the disease is prostate cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrdrogenase level, neutrophil to lymphocyte ratio, number of bone metastases, liver metastases present, PSA level, years since original PCA diagnosis, prior 2^{nd} generation novel hormonal therapy, prior taxane, small cell histology, PSA doubling time/PSA velocity, recent development of new lesions, CTC count, ctDNA fraction, bone alkaline phosphatase level, presence of N-telopeptides in urine, extent of bone involvement, patient mobility, patient ability to climb stairs, insulin use, antithrombotic use, antiarrhythmic agent use, or any combination thereof.
15. The computer-implemented method of clause 3, wherein the disease is renal cell carcinoma and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, histology, disease stage at diagnosis, International Metastatic RCC Database Consortium (IMDC) risk score, recent diagnosis of metastases, recurrence of metastases within 1 year vs recurrence after 1 year, hypercalcemia, neutrophil status, platelet status, anemia, c-reactive protein level, inflammation, IL-6 level, IL-8 level, HGF hepatocyte growth factor level, osteopontin level, BAP1 level, PBRM1 level, 3p loss, 5q gain, 7q gain, 8p loss, 9p loss, 14q loss, or any combination thereof.
16. The computer-implemented method of clause 3, wherein the disease is small cell lung cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, SCLC stage at diagnosis, disease stage at diagnosis, smoking status, brain metastases, localized symptomatic sites, resection status, radiotherapy status, prior immunotherapy status, or any combination thereof.
17. The computer-implemented method of clause 3, wherein the disease is urothelial cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, primary site, disease stage at diagnosis, disease grade at diagnosis, CBC, asparate aminotransferase level, alanine aminotransferase level, bilirubin level, glomerular filtration rate, PD-L1 status, fibroblast growth factor receptor (FGFR) status, De Ritis ratio, or any combination thereof.
18. The computer-implemented method of any one of clauses 1 to 17, wherein the data in the second data set that is derived from data in the first data set comprises a patient's age at a start of a line of therapy, a determination of whether the patient's albumin levels are less than a lower limit of normal, a determination of whether the patient's alkaline phosphatase levels are greater than an upper limit of normal, a determination of whether the patient's serum creatinine levels are greater than an upper limit of normal, a determination of the patient's pooled ECOG value, a determination of whether the patient should be excluded when applying a 90 day gap rule, a determination of whether the patient's hemoglobin levels are less than a lower limit of normal, a determination of whether the patient's line of therapy has had a maintenance line rolled in, a determination of whether the patient's lactate dehydrogenase levels are greater than an upper limit of normal, a determination of a numerical value for a neutrophil-to-lymphocyte ratio, a determination of whether the neutrophil-to-lymphocyte ratio is greater than 2.5, a determination of whether the patient has evidence of having received opioid pain medication in a period of 62 days preceding the start of the line of therapy, a determination of an end date used in a calculation of the patient's overall survival (OS), a determination of a time to death or censoring for the patient's OS analysis, a determination of an entry date used in the calculation of the patient's OS, a determination of the patient's delayed entry time in months, a determination of whether the end date was an event or censor for OS analysis, a determination of whether the patient has evidence of having received steroid medication in a period of 62 days preceding the start date of their line of therapy, a determination of whether the patient's time to discontinuation (TTD) was an event or censor, a determination of TTD in months for TTD analysis, a determination of whether the patient's time to next treatment (TTNT) was an event or censor, a determination of TTNT in months for TTNT analysis, disease-free survival (DFS), time-to-treatment failure (TTF), durable complete response (DCR), or any combination thereof.
19. The computer-implemented method of any one of clauses 1 to 18, wherein the data in the second data set that is derived from data in the first data set comprises a pre-computed endpoint for a survival analysis or a time-to-event analysis.
20. The computer-implemented method of clause 19, wherein the pre-computed endpoint for survival analysis comprises time from start of any line of treatment of the disease to a time of death, overall survival (OS), progression free survival (PFS), objective response rate (ORR), time-to-tumor progression (TTP), time-to-next treatment (TTNT), disease-free survival (DFS), time-to-tumor progression (TTP), time-to-treatment failure (TTF), or durable complete response (DCR).
21. The computer-implemented method of any one of clauses 1 to 20, wherein the standardized table format comprises a matrix in which each row specifies a line of therapy, and each column comprises data for a category of prognostic and analytically useful variable.
22. The computer-implemented method of any one of clauses 1 to 21, wherein the clinico-genomic database can comprise a single entity or a joint collaboration clinico-genomic database (CGDB).
23. A computer-implemented method for performing an analysis of clinico-genomic data comprising:

receiving, at one or more processors, a first input from a user, wherein the first input specifies a disease;

accessing, using the one or more processors, a standardized table of clinico-genomic data for the disease;

receiving, at the one or more processors, at least a second input from the user;

performing, using the one or more processors, an analysis based on the at least second input and the clinico-genomic data included in the standardized table; and

outputting, using the one or more processors, a result of the analysis of the clinico-genomic data included in the standardized table.
24. The computer-implemented method of clause 23, wherein the method further comprises accessing additional data from a clinico-genomic database and using the additional data as part of the analysis.
25. The computer-implemented method of clause 23 or clause 24, wherein the analysis comprises a Kaplan Meier survival analysis or a log rank test.
26. The computer-implemented method of clause 23 or clause 24, wherein the analysis comprises a statistical regression analysis.
27. The computer-implemented method of clause 26, wherein the statistical regression analysis comprises a Cox proportional hazards regression analysis.
28. A system comprising:

one or more processors; and

a memory communicatively coupled to the one or more processors and configured to store instructions that, when executed by the one or more processors, cause the system to:

receive, at one or more processors, input data that specifies a plurality of prognostic features for a disease;

extract, using the one or more processors, a first data set comprising data corresponding to the plurality of prognostic features for the disease from a clinico-genomic database;

generate, using the one or more processors, a second data set based on the first data set, wherein the second data set comprises data from the first data set, data derived from data in the first data set, or a combination thereof; and

store, using the one or more processors, the second data set in a standardized table format.
29. A non-transitory computer-readable storage medium storing one or more programs, the one or more programs comprising instructions, which when executed by one or more processors of a system, cause the system to:

receive, at one or more processors, input data that specifies a plurality of prognostic features for a disease;

extract, using the one or more processors, a first data set comprising data corresponding to the plurality of prognostic features for the disease from a clinico-genomic database;

generate, using the one or more processors, a second data set based on the first data set, wherein the second data set comprises data from the first data set, data derived from data in the first data set, or a combination thereof; and

store, using the one or more processors, the second data set in a standardized table format.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A computer-implemented method for processing clinico-genomic data comprising:
receiving, at one or more processors, input data that specifies a plurality of prognostic features for a disease;
extracting, using the one or more processors, a first data set comprising data corresponding to the plurality of prognostic features for the disease from a clinico-genomic database;
generating, using the one or more processors, a second data set based on the first data set, wherein the second data set comprises data from the first data set, data derived from data in the first data set, or a combination thereof;
storing, using the one or more processors, the second data set in a standardized table format; and
outputting the second data set in the standardized table format.

2. (canceled)

3. The computer-implemented method of claim 1, wherein the disease is breast cancer, colorectal cancer, endometrial cancer, gastric cancer, hepatocellular carcinoma, head and neck cancers, melanoma, non-small-cell lung cancer, ovarian cancer, pancreatic cancer, prostate cancer, renal cell carcinoma, small cell lung cancer, or urothelial cancer.

4. The computer-implemented method of claim 3, wherein the disease is breast cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, histology, presence of brain metastases, bone metastases, stage of diagnosis, menopausal status, visceral crisis, ER status, PR status, HER2 status, PD-L1 immunohistochemistry, germline BRCA status, PI3CA status, or any combination thereof.

5. The computer-implemented method of claim 3, wherein the disease is colorectal cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, colorectal site (including sidedness for colon cancer), stage of diagnosis, BRAF mutation status, RAS mutation status, dMMR/MSI, HER2 status, consensus molecular subtypes, platelets status, or any combination thereof.

6. The computer-implemented method of claim 3, wherein the disease is endometrial cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrodgenase level, neutrophil to lymphocyte ratio, histology, grade, ER status, PR status, HER2 status, TCGA subgroup, POLE status, MSI-H/dMMR status, TP53 status, presence of brain metastases, presence of metastases above a diaphragm, disease stage at diagnosis, beta-catenin alteration status, serum CA-125 level, history of endometriosis, BMI, residual disease in abdomen after primary surgery, blood pressure, or any combination thereof.

7. The computer-implemented method of claim 3, wherein the disease is gastric cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, tumor type/disease site, disease stage at diagnosis, Siewert classification, smoking status, anemia, H. pylori status, alcohol use, EBV, surgery, HER2 status, PD-L1 status, MSI/MMR status, family history, or any combination thereof.

8. The computer-implemented method of claim 3, wherein the disease is hepatocellular carcinoma, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, disease stage at diagnosis, Child-Pugh score, encephalopathy, ascites, bilirubin status, primary biliary cirrhosis status, aspartate transaminase status, alanine transaminase status, albumin (quantitative), prothrombin time (PT), international normalized ratio (INR), blood urea nitrogen (BUN), complete blood count (CBC), platelets status, alpha fetoprotein (AFP) status, ALBI grade, microsatellite instability (MSI) status, mismatch repair (MMR) status, tumor mutational burden (TMB), or any combination thereof.

9. The computer-implemented method of claim 3, wherein the disease is a head and neck cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, primary site, disease stage at diagnosis, smoking status, HPV/p16 status, alcohol use, Epstein-Barr virus (EBV) status, surgery status, radiotherapy status, PD-L1 status, or any combination thereof.

10. The computer-implemented method of claim 3, wherein the disease is melanoma, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, sites of metastases, BRAF V600 mutation status, KIT status, NRAS status, NTRK status, Breslow thickness, ulceration, mitotic rate, tumor location (axial vs extremity), lymphovascular invasion, microsatellites (local spread), NF1, prednisone, or any combination thereof.

11. The computer-implemented method of claim 3, wherein the disease is non-small-cell lung cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehyrdrogenase level, neutrophil to lymphocyte ratio, histology, smoking history, presence of brain metastases, bone metastases, disease stage at diagnosis, EGFR mutation status, ALK rearrangement status, ROS1 rearrangement status, BRAF mutation status, KRAS mutation status, MET exon 14 skipping mutation status, RET rearrangement status, NTRK rearrangement status, MET amplification status, ERBB2 mutation status, PD-L1 immunohistochemistry, TMB, or any combination thereof.

12. The computer-implemented method of claim 3, wherein the disease is ovarian cancer, and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, histology, disease stage at diagnosis, disease grade at diagnosis, HRD status, CA-125 status, TP53 status, gabapentin status, surgical removal of macroscopic disease (R0) status, platinum sensitivity, neoadjuvant chemotherapy vs surgery as an initial intervention, family history, germline BRCA status, BRCA1 status, BRCA2 status, RAD51C status, RAD51D status, BARD1 status, BRIP1status, PALB2 status, MLH1 status, MSH2 status, MSH6 status, PMS2 status, STK11 status, or any combination thereof.

13. The computer-implemented method of claim 3, wherein the disease is pancreatic cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, high neutrophil to lymphocyte ratio, disease stage at diagnosis, radiotherapy, cancer antigen 19-9 status, MSI status, MMR status, TMB, prior therapies, BRCA mutation status, PALB2 mutation status, ALK fusion status, NRG1 fusion status, NTRK fusion status, ROS1 fusion status, BRAF mutation status, HER2 mutation status, KRAS mutation status, or any combination thereof.

14. The computer-implemented method of claim 3, wherein the disease is prostate cancer and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, number of bone metastases, liver metastases present, PSA level, years since original PCA diagnosis, prior 2^{nd} generation novel hormonal therapy, prior taxane, small cell histology, PSA doubling time/PSA velocity, recent development of new lesions, CTC count, ctDNA fraction, bone alkaline phosphatase level, presence of N-telopeptides in urine, extent of bone involvement, patient mobility, patient ability to climb stairs, insulin use, antithrombotic use, antiarrhythmic agent use, or any combination thereof.

15. The computer-implemented method of claim 3, wherein the disease is renal cell carcinoma and the plurality of prognostic features comprises a line of therapy, practice type, sex, race, insurance status, age, ECOG performance status, pre-therapy opioid pain medication use, pre-therapy (cortico)steroid use, albumin level, alkaline phosphatase level, creatinine level, hemoglobin level, lactate dehydrogenase level, neutrophil to lymphocyte ratio, histology, disease stage at diagnosis, International Metastatic RCC Database Consortium (IMDC) risk score, recent diagnosis of metastases, recurrence of metastases within 1 year vs recurrence after 1 year, hypercalcemia, neutrophil status, platelet status, anemia, c-reactive protein level, inflammation, IL-6 level, IL-8 level, HGF hepatocyte growth factor level, osteopontin level, BAP1 level, PBRM1 level, 3p loss, 5q gain, 7q gain, 8p loss, 9p loss, 14q loss, or any combination thereof.

16. (canceled)

17. (canceled)

18. The computer-implemented method of claim 1, wherein the data in the second data set that is derived from data in the first data set comprises a patient's age at a start of a line of therapy, a determination of whether the patient's albumin levels are less than a lower limit of normal, a determination of whether the patient's alkaline phosphatase levels are greater than an upper limit of normal, a determination of whether the patient's serum creatinine levels are greater than an upper limit of normal, a determination of the patient's pooled ECOG value, a determination of whether the patient should be excluded when applying a 90 day gap rule, a determination of whether the patient's hemoglobin levels are less than a lower limit of normal, a determination of whether the patient's line of therapy has had a maintenance line rolled in, a determination of whether the patient's lactate dehydrogenase levels are greater than an upper limit of normal, a determination of a numerical value for a neutrophil-to-lymphocyte ratio, a determination of whether the neutrophil-to-lymphocyte ratio is greater than 2.5, a determination of whether the patient has evidence of having received opioid pain medication in a period of 62 days preceding the start of the line of therapy, a determination of an end date used in a calculation of the patient's overall survival (OS), a determination of a time to death or censoring for the patient's OS analysis, a determination of an entry date used in the calculation of the patient's OS, a determination of the patient's delayed entry time in months, a determination of whether the end date was an event or censor for OS analysis, a determination of whether the patient has evidence of having received steroid medication in a period of 62 days preceding the start date of their line of therapy, a determination of whether the patient's time to discontinuation (TTD) was an event or censor, a determination of TTD in months for TTD analysis, a determination of whether the patient's time to next treatment (TTNT) was an event or censor, a determination of TTNT in months for TTNT analysis, disease-free survival (DFS), time-to-treatment failure (TTF), durable complete response (DCR), or any combination thereof.

19. The computer-implemented method of claim 1, wherein the data in the second data set that is derived from data in the first data set comprises a pre-computed endpoint for a survival analysis or a time-to-event analysis.

20. (canceled)

21. A computer-implemented method for performing an analysis of clinico-genomic data comprising:
receiving, at one or more processors, a first input from a user, wherein the first input specifies a disease;
accessing, using the one or more processors, a standardized table of clinico-genomic data for the disease;
receiving, at the one or more processors, at least a second input from the user;
performing, using the one or more processors, an analysis based on the at least second input and the clinico-genomic data included in the standardized table; and
outputting, using the one or more processors, a result of the analysis of the clinico-genomic data included in the standardized table.

22. (canceled)

23. The computer-implemented method of claim 21, wherein the analysis comprises a Kaplan Meier survival analysis or a log rank test.

24. The computer-implemented method of claim 21, wherein the analysis comprises a statistical regression analysis.

25. (canceled)

26. A system comprising:
one or more processors; and
a memory communicatively coupled to the one or more processors and configured to store instructions that, when executed by the one or more processors, cause the system to:
receive input data that specifies a plurality of prognostic features for a disease;
extract a first data set comprising data corresponding to the plurality of prognostic features for the disease from a clinico-genomic database;
generate a second data set based on the first data set, wherein the second data set comprises data from the first data set, data derived from data in the first data set, or a combination thereof; and store the second data set in a standardized table format.
